(19) United States
(12) Patent Application Publication
(10) Pub. No.: US 2007/0135448 A1
(43) Pub. Date: Jun. 14, 2007

(54) Use of cathepsin k inhibitors for treating of severe bone loss diseases

(76) Inventors: Martin Missbach, Gipf-Oberfrick (CH); Rainer Gamse, Basel (CH); Ulrich Trechsel, Luzein (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/578,167

(22) PCT Filed: Apr. 19, 2004
(86) PCT No.: PCT/EP04/04155

§ 371 Date: May 4, 2006

(30) Foreign Application Priority Data
Nov. 19, 2003 (EP) 03026430.3

(51) Int. Cl. A61K 31/495; A61K 31/41; A61K 31/4196; A61K 31/4172

(52) U.S. Cl. 514/255.01; 514/381; 514/383; 514/400

(57) ABSTRACT

This invention relates generally to cathepsin K inhibitors and their use in bone growth. Specifically, the invention relates to the use of cathepsin K inhibitors to stimulate new bone formation in patients in need thereof.

This invention relates generally to cathepsin K inhibitors and their use in bone growth. Specifically, the invention relates to the use of cathepsin K inhibitors to stimulate new bone formation in patients in need thereof.

Cathepsin K was cloned and found specifically expressed in osteoclasts (Tezuka, K. et al., 1994, J Biol Chem 269:1106-1109; Shi, G. P. et al., 1995, FEBS Lett 357:129-134; Bromme, D. and Okamoto. K., 1995, Biol Chem Hoppe Seyler 376:379-384-, Bromme, D. et al., 1996, J Biol Chem 271:2126-2132; Drake, F. H. et al., 1996, J Biol Chem 27 1: 12511-12516). Concurrent to the cloning, the autosomal recessive disorder, pycnodysostosis, characterized by an osteopetrotic phenotype with a decrease in bone resorption, was mapped to mutations present in the cathepsin K gene. To date, all mutations identified in the cathepsin K gene are known to result in inactive protein (Gelb, B. D. et al., 1996, Science 273:1236-1213) 8: Johnson, M. R. et al., 1996, Genome Res 6:1050-1055. Cathepsin K is synthesized as a '37 kDa pre-pro enzyme, which is localized to the lysosomal compartment and where it is presumably autoactivated to the mature 27 kDa enzyme at low pH (McQueney, M. S. et al., 1997, Biol Chem 272:13955-13960; Littlewood-Evans, A. et al., 1997, Bone 20:81-86). Cathepsin K is most closely related to cathepsin S having 56% sequence identity at the amino acid level. The S2P2 substrate specificity of cathepsin K is similar to that of cathepsin S with a preference in the P1 and P2 positions for a positively charged residue such as arginine, and a hydrophobic residue such as phenylalanine or leucine, respectively (Bromme, D. et al., 1996, J Biol Chem 271: 2126-2132; Bossard, M. J. et al., 1996) J Biol Chem 271:12517-12524). Cathepsin K is active at a broad pH range with significant activity between pH 4-8, thus allowing for good catalytic activity in the resorption lacunae of osteoclasts where the pH is about 4-5. Human type I collagen, the major collagen in bone is a good substrate for cathepsin K (Kafienah, W., et al., 1998, Biochem J 331:727-732). In vitro experiments using antisense oligonucleotides to cathepsin K, have shown diminished bone resorption in vitro probably due to a reduction in translation of cathepsin K mRNA (Inui, T., et al., 1997, J Biol Chem 272:8109-8112. The crystal structure of cathepsin K has been resolved (McGrath, M. E., et al., 1997, Nat Struct Biol 4:105-109; Zhao, B., et al., 1997, Nat Struct Biol 4: 109-11) and selective peptide based inhibitors of cathepsin K (Bromme, D., et al., 1996, Biochem J 315:85-89-, Thompson, S. K., et al.,1997, Proc Natl Acad Sci USA 94:14249-14254) and non-peptide based inhibitors of cathepsin K (WO 03/020721) have been developed. Bone resorption, is primarily performed by multi nuclear giant cells, the osteoclasts. The mechanism by which osteoclasts resorb bone is by an initial cellular attachment to bone tissue followed by the formation of an extracellular compartment or lacunae. The lacunae are maintained at a low pH by a proton-ATP pump. The acidified environment allows for initial demineralization of bone followed by the degradation of bone proteins or collagen by proteases such as cysteine proteases (Delaisse, J. M. et al., 1980, Biochem J 192:365-368; Delaisse, J. et al., 1984, Biochem Biophys Res Commun:441-447; Delaisse, J. M. et al., 1987, Bone 8:305-313). Collagen constitutes 95% of the organic matrix of bone. Therefore, proteases such as cathepsin K involved in collagen degradation are an essential component of bone turnover. The skeleton is constantly being remodeled by a balance between osteoblasts that lay down new bone and osteoclasts that break down, or resorb bone. In some disease conditions and advancing age the balance between bone formation and resorption is disrupted; bone is removed at a faster rate. Such a prolonged imbalance of resorption over a long duration leads to weaker bone structure and a higher risk of fractures.

In accordance with the present invention, it has now surprisingly been found that cathepsin K inhibitors exert bone forming effects in an in vivo animal model (see Example 1). For example, a bone forming effect on certain bones, e.g. increased bone mineral density (BMD) and increased bone strength is observed when a cathepsin K inhibitor is administered orally to ovariectomized (OVX) cynomolgus monkeys twice daily for eighteen months.

Thus, cathepsin K inhibitors are particularly useful in the treatment of a severe form of various bone loss disorders, including e.g. osteoporosis, osteopenia, tumors (especially tumor invasion and bone metastases (BM)), tumor-induced hypercalcemia (TIH) and multiple myeloma (MM).

Accordingly, the present invention provides a method for the treatment of a severe form of bone loss diseases in a patient in need of such treatment, which comprises administering an effective amount of a cathepsin K inhibitor to the patient.

The invention further provides the use of a cathepsin K inhibitor in the preparation of a medicament for the treatment of a severe form of bone loss diseases in mammals, e.g. humans.

The invention yet further provides the use of a cathepsin K inhibitor and other agents, useful in the treatment of bone loss diseases, to treat a severe form of bone loss diseases in mammals, e.g. humans.

Preferably the invention is used for the treatment of diseases and medical conditions in which cathepsin K inhibitors are used to stimulate bone growth. For example, the invention may be used for the treatment of diseases and conditions which involve excessive or inappropriate bone loss e.g. as the result of inappropriate bone metabolism. Examples of such diseases and conditions include severe forms of benign diseases and conditions such as osteoporosis of various genesis, periodontal disease; and especially malignant diseases such as MM and TIH and BM associated with various cancers, e.g. cancer of the breast, prostate, lung, kidney, ovary, or osteosarcoma. Generally the invention may be used to treat severe bone loss diseases also in other circumstances where cathepsin K inhibitors may be used, e.g. when cathepsin K inhibitors are used in bone fracture healing, osteonecrosis or treatment of prosthesis loosening. Cathepsin K inhibitors are particularly useful for treating severe forms of diseases of bone metabolism including osteoporosis, osteoarthritis, and other inflammatory arthritides, and bone loss in general, including age-related bone loss, and in particular periodontal disease.

Furthermore, cathepsin K inhibitors surprisingly improve bone strength due not only through their anti-resorptive effect (which is expected and known from the literature) but also through their surprising bone-forming effect. Preferably cathepsin K inhibitors increase spinal and femoral bone mineral density (BMD) and bone strength.

Thus, the invention relates to the use of cathepsin K inhibitors for the manufacture of a medicament for reducing the risk of bone fracture, preferably spinal and femoral bone fracture, in mammals, preferably a mammal, e.g. human, more preferably a post menopausal woman at risk of or having osteoporosis, e.g. severe osteoporosis. The medicament can be employed to increase stiffness and/or toughness at a site of a potential trauma or at a site of an actual trauma. Trauma generally includes fracture, surgical trauma, joint replacement, orthopaedic procedures, and the like. Increasing bone toughness and/or stiffness generally includes increasing mineral density of particular bones, e.g. the subperiosteal site of the vertebrae and long bones, increasing strength of bone, and the like. Reducing incidence of fracture generally includes reducing the likelihood or actual incidence of fracture for a subject compared to an untreated control population. Moreover, femoral bone mineral density can predict the long-term risk for bone fracture in general (Melton et al, J. of Bone and Miner Res, 2003; 18(2):312-318).

The uses and methods of the present invention represent an improvement to existing therapy of bone loss diseases in which e.g. bisphosphonates are used to prevent or inhibit development of bone metastases or excessive bone resorption, and also for the therapy of inflammatory diseases such as rheumatoid arthritis and osteoarthritis, as well as for all forms of osteoporosis and osteopenia.

Thus in the present description the terms “treatment” or “treat” refer to both prophylactic or preventative treatment as well as curative or treatment of severe bone loss diseases, in particular treatment of severe osteoporosis.

Thus in particular embodiments the invention provides: a method for the treatment of a severe form of bone loss disease in a patient in need of such treatment which comprises administering an effective amount of a cathepsin K inhibitor to the patient; the use of a cathepsin K inhibitor in the preparation of a medicament for the treatment of a severe form or severe forms of bone loss diseases; or the use of a cathepsin K inhibitor as an agent for treatment of a severe form or severe forms of bone loss diseases.

For these indications, the appropriate dosage will, of course, vary depending upon, for example, the particular cathepsin K inhibitor to be employed, the host, the mode of administration and the nature and severity of the condition being treated. However, in general, satisfactory results in animals are indicated to be obtained at a daily dosage from about 1 to about 300 mg/kg animal body weight. In larger mammals, for example humans, an indicated daily dosage is in the range from about 0.01 to about 2 g of a compound according to the invention, conveniently administered, for example, in divided doses up to four times a day. The cathepsin K inhibitors may be administered in any usual manner, e.g. orally, for example in the form of tablets or capsules, or parenterally, for example in the form of injection solutions or solutions.

The present invention also provides pharmaceutical compositions comprising the cathepsin K inhibitors in association with at least one pharmaceutical carrier or diluent for use in the treatment of a severe form of bone loss diseases. Such compositions maybe manufactured in conventional manner. Unit dosage forms may contain for example from about 2.5 mg to about 1000 mg of the cathepsin K inhibitor.

In another aspect, the invention provides particular a dosage range for a specific cathepsin K inhibitor, i.e. N-[1-(cyanomethyl-carbamoyl)-cyclohexyl]-4-(4-propyl-piperazin-1-yl)-benzamide (Compound A), which is efficacious and well tolerated, i.e. safe for a patient to take. Preferred is a range between 1 and 50 mg Compound A or a pharmaceutically acceptable salt thereof wherein the amount of the base of Compound A is between 1 and 50 mg per day for an adult person, i.e. a person older than 20 years. More preferred are dosages below 50.1 mg of Compound A or a pharmaceutically acceptable salt wherein the amount of the base of Compound A is less than 50.1 mg, e.g. 1 mg, 5 mg, 10 mg, 25 mg, 50 mg; even more preferred between 1 and 50 mg, e.g. 1 mg, 5 mg, 10 mg, 25mg, 50 mg; even more preferred between 5 and 50 mg, e.g. 5 mg, 10 mg, 25 mg, 50 mg; even more preferred between 5 and 25 mg, e.g. 5 mg, 10 mg, 25 mg or other preferred dosages are 1, 5, 10, 25 or 50 mg of Compound A or a pharmaceutically acceptable salt thereof wherein the amount of the base of Compound A is 1, 5, 10, 25 or 50 mg. A preferred salt for Compound A is the maleate salt. E.g. a preferred range is between 1 and 64.1 mg of the maleate salt of Compound A, e.g. less than 64.2 mg.

The cathepsin K inhibitors used in the present invention are typically those which form bone, in particular stimulate cortical bone formation at subperiosteal site, i.e. vertebrae and long bones. Preferably, the cathepsin K inhibitors used in the pharmaceutical compositions and treatment methods of the present invention typically comprises a cathepsin K inhibitor, e.g. disclosed in WO 9523222, WO 9630353, WO 9640737, WO 9716433, WO 9801133, WO 9805336, WO 9808802, WO 9846582, WO 9848799, WO 9849152, WO 9850342, WO 9850533, WO 9850534, WO 9911637, WO 9924460, WO 9948911, WO 9959526, WO 9959570, WO 9964399, WO 9966925, WO 0029408, WO 0038687, WO 0039115, WO 0048993, WO 0049011, WO 0054769, WO 0055124, WO 0055125, WO 0055126, WO 0055144, WO 0058296, WO 0059881, WO 0109110, WO 0109169, WO 0119808, WO 0119816, WO 0134153, WO 0134154, WO 0134155, WO 0134156, WO 0134157, WO 0134158, WO 0320721, WO 0320278, WO 0313518, WO 02100849, WO 0298406, WO 0296892, WO 0292563, WO 0288106, WO 0280920, WO 0270519, WO 0270517, WO 0269992, WO 0269901, WO 0257270, WO 0257249, WO 0257248, WO 0257246, WO 0158886, WO 0155123 or a compound of formula V, or a physiologically acceptable and—cleavable ester or a salt thereof 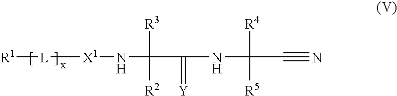
wherein R¹ is optionally substituted (aryl, aryl-lower alkyl, lower alkenyl, lower alkynyl, heterocyclyl or heterocyclyl-lower alkyl);
R² and R³ together represent lower alkylene, optionally interrupted by O, S or NR⁶, so as to form a ring with the carbon atom to which they are attached, and R⁶ is hydrogen, lower alkyl or aryl-lower alkyl;
R⁴ and R⁵ are independently H, or optionally substituted (lower alkyl or aryl-lower alkyl), —C(O)OR⁷, or —C(O)NR⁷R⁸, wherein R⁷ is optionally substituted (lower alkyl, aryl, aryl-lower alkyl, cycloalkyl, bicycloalkyl, bicycloalkyl or heterocyclyl), and R⁸ is H, or optionally substituted (lower alkyl, aryl, aryl-lower alkyl, cycloalkyl, bicycloalkyl, bicycloalkyl or heterocyclyl); or
R⁴ and R⁵ together represent lower alkylene, optionally interrupted by O, S or NR⁶, so as to form a ring with the carbon atom to which they are attached, and R⁶ is hydrogen, lower alkyl or aryl-lower alkyl; or
R⁴ is H or optionally substituted lower alkyl and R⁵ is a substituent of formula —X²—(Y¹)ₙ—(Ar)ₚ-Q-Z wherein
Y¹ is O, S, SO, SO₂, N(R⁶)SO₂, N—R⁶, SO₂NR⁶, CONR⁶ or NR⁶CO;
N is zero or one;
P is zero or one;
X² is lower alkylene: or when n is zero, X² is also C₂-C₇-alkylene interrupted by O, S, SO, SO₂, NR⁶, SO₂NR⁶, CONR⁶ or NR⁶CO, and R⁶ is hydrogen, lower alkyl or aryl-lower alkyl;
Ar is arylene;
Z is hydroxyl, acyloxy, carboxyl, esterified carboxyl, amidated carboxyl, aminosulfonyl, (lower alkyl or aryl-lower alkyl)aminosulfonyl, or (lower alkyl or aryl-lower alkyl)sufonylaminocarbonyl; or Z is tetrazolyl, triazolyl or imidazolyl;
Q is a direct bond, lower alkylene, Y¹-lower alkylene or C₂-C₇-alkylene interrupted by Y¹;
X¹ is —C(O)—, —C(S)—, —S(O)—, —S(O)₂-, or —P(O)(OR⁶)-, and R⁶ is as defined above;
Y is oxygen or sulphur;
L is optionally substituted -Het-, -Het-CH₂- or —CH₂-Het-, and Het is a hetero atom selected from O, N or S; and
X is zero or one; and
aryl in the above definitions represents carbocyclic or heterocyclic aryl.

Particular compounds of formula V are those wherein R¹ is a substituted phenyl, e.g. whereas the substituent is an optionally substituted nitrogen-containing heterocyclic substituent (=Het^{IV}). This substituent may be at the 2- or 3- position of the phenyl ring, though preferably at the 4-position. Het^{IV} signifies a heterocyclic ring system containing at least one nitrogen atom, from 2 to 10, preferably from 3 to 7, most preferably 4 or 5, carbon atoms and optionally one or more additional heteroatoms selected from O, S or preferably N.

Het^{IV} may comprise an unsaturated, e.g. an aromatic, nitrogen-containing heterocycle; though preferably comprises a saturated nitrogen-containing heterocycle. Particularly preferred saturated nitrogen-containing heterocycles are piperazinyl, preferably piperazin-1-yl, or piperidinyl, preferably piperidin-4-yl.

Het^{IV} may be substituted by one or more substituents, e.g. by up to 5 substituents independently selected from halogen, hydroxy, amino, nitro, optionally substituted C₁₋₄alkyl (e.g. alkyl substituted by hydroxy, alkyloxy, amino, optionally substituted alkylamino, optionally substituted dialkylamino, aryl or heterocyclyl), C₁₋₄alkoxy. Preferably Het^{IV} is substituted at a nitrogen atom, most preferably mono-substituted at a nitrogen atom. Preferred substituents for Het^{IV} are C₁-C₇lower alkyl, C₁-C₇lower alkoxy-C₁-C₇lower alkyl, C₅-C₁₀aryl-C₁-C₇lower alkyl, or C₃-C₈cycloalkyl.

Particularly preferred embodiments of the invention provides a compound of formula VI, or a pharmaceutically acceptable salt or ester thereof 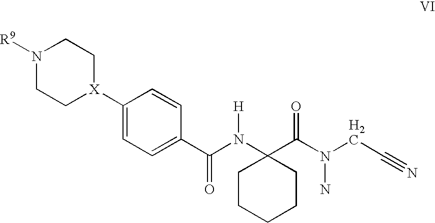
wherein X is CH or N, and
R⁹ is H, C₁-C₇lower alkyl, C₁-C₇lower alkoxy-C₁-C₇lower alkyl, C₅-C₁₀aryl-C₁-C₇lower alkyl, or C₃-C₈cycloalkyl.

Thus particular examples of R⁹ as C₁-C₇lower alkyl are methyl, ethyl, n-propyl, or i-propyl are preferred. A particular example of R as C₁-C₇lower alkoxy-C₁-C₇lower alkyl is methoxyethyl. A particular example of R as C₅-C₁₀aryl-C₁-C₇lower alkyl is benzyl. A particular example of R as C₃-C₈cycloalkyl is cyclopentyl. Examples of particular compounds of formula VI are: N-[1-(Cyanomethyl-carbamoyl)-cyclohexyl]-4-(piperazin-1-yl)-benzamide; N-[1-(Cyanomethyl-carbamoyl)-cyclohexyl]-4-(4-methyl-piperazin-1-yl)-benzamide; N-[1-(Cyanomethyl-carbamoyl)-cyclohexyl]-4-(4-ethyl-piperazin-1-yl)-benzamide; N-[1-(Cyanomethyl-carbamoyl)-cyclohexyl]-4-[4-(1-propyl)-piperazin-1-yl]-benzamide; N-[1-(Cyanomethyl-carbamoyl)-cyclohexyl]-4-(4-isopropyl-piperazin-1-yl)-benzamide; N-[1-(Cyanomethyl-carbamoyl)-cyclohexyl]-4-(4-benzyl-piperazin-1-yl)-benzamide; N-[1-(Cyanomethyl-carbamoyl)-cyclohexyl]-4-[4-(2-methoxy-ethyl)-piperazin-1-yl]-benzamide; N-[1-(Cyanomethyl-carbamoyl)-cyclohexyl]-4-(1-propyl-piperidin-4-yl)-benzamide; N-[1-(Cyanomethyl-carbamoyl)-cyclohexyl]-4-[1-(2-methoxy-ethyl)-piperidin-4-yl]-benzamide; N-[1-(Cyanomethyl-carbamoyl)-cyclohexyl]-4-(1-isopropyl-piperidin-4-yl)-benzamide; N-[1-(Cyanomethyl-carbamoyl)-cyclohexyl]-4-(1-cyclopentyl -piperidin-4-yl)-benzamide; N-[1-(Cyanomethyl-carbamoyl)-cyclohexyl]-4-(1-methyl-piperidin-4-yl)-benzamide, and N-[1-(Cyanomethyl-carbamoyl)-cyclohexyl]-4-(piperidin-4-yl)-benzamide.

The most preferred cathepsin K inhibitor for use in the invention is N-[1-(Cyanomethyl-carbamoyl)-cyclohexyl]-4-[4-(1-propyl)-piperazin-1-yl]-benzamide or a pharmacologically acceptable salt thereof.

All the cathepsin K inhibitors mentioned above are known from the literature. This includes their production (see e.g. U.S. Pat. No. 6,353,017B1, pp. 15-17).
An alternative class of cathepsin K inhibitors compounds for use in the invention comprises a compound of formula VII, or a physiologically acceptable and -cleavable ester or a salt thereof 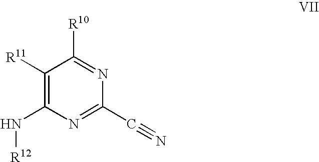
wherein
R¹⁰ is H, —R¹⁴, —OR¹⁴ or NR¹³R¹⁴,
wherein R¹³ is H, lower alkyl or C₃ to C₁₀ cycloalkyl, and
R¹⁴ is lower alkyl or C₃ to C₁₀ cycloalkyl, and
wherein R¹³ and R¹⁴ are independently, optionally substituted by halo, hydroxy, lower alkoxy, CN, NO₂, or optionally mono- or di-lower alkyl substituted amino;
R¹¹ is —CO—N R¹⁵ R¹⁶, —NH—CO—R¹⁵, —CH₂—NH—C(O)—R¹⁵, —CO—R¹⁵, —S(O)—R¹⁵, —S(O)₂—R¹⁵, —CH₂—CO—R¹⁵ or —CH₂—NR¹⁵R¹⁶,
wherein
R¹⁵ is aryl, aryl-lower alkyl, C₃-C₁₀cycloalkyl, C₃-C₁₀cycloalkyl-lower alkyl, heterocyclyl or heterocyclyl-lower alkyl,
R¹⁶ is H, aryl, aryl-lower alkyl, aryl-lower-alkenyl, C₃-C₁₀cycloalkyl, C₃-C₁₀cycloalkyl-lower alkyl, heterocyclyl or heterocyclyl-lower alkyl, or
wherein R¹⁵ and R¹⁶ together with the nitrogen atom to which they attached are joined to form an N-heterocyclyl group,
wherein N-heterocyclyl denotes a saturated, partially unsaturated or aromatic nitrogen containing heterocyclic moiety attached via a nitrogen atom thereof having from 3 to 8 ring atoms optionally containing a further 1, 2 or 3 heteroatoms selected from N, NR¹⁷, O, S, S(O) or S(O)₂ wherein R¹⁷ is H or optionally substituted (lower alkyl, carboxy, acyl (including both lower alkyl acyl, e.g. formyl, acetyl or propionyl, or aryl acyl, e.g. benzoyl), amido, aryl, S(O) or S(O)₂), and wherein the N-heterocyclyl is optionally fused in a bicyclic structure, e.g. with a benzene or pyridine ring, and wherein the N-heterocyclyl is optionally linked in a spiro structure with a 3 to 8 membered cycloalkyl or heterocyclic ring wherein the heterocyclic ring has from 3 to 10 ring members and contains from 1 to 3 heteroatoms selected from N, NR¹⁶, O, S, S(O) or S(O)₂ wherein R¹⁶ is as defined above), and
wherein heterocyclyl denotes a ring having from 3 to 10 ring members and containing from 1 to 3 heteroatoms selected from N, NR¹⁷, O, S, S(O) or S(O)₂ wherein R¹⁷ is as defined above), and wherein R¹⁵ and R¹⁶ are independently, optionally substituted by one or more groups, e.g. 1-3 groups, selected from halo, hydroxy, oxo, lower alkoxy, CN or NO₂, or optionally substituted (optionally mono- or di-lower alkyl substituted amino, lower-alkoxy, aryl, aryl-lower alkyl, N-heterocyclyl or N-heterocyclyl-lower alkyl (wherein the optional substitution comprises from 1 to 3 substituents selected from halo, hydroxy, lower alkoxy, lower alkoxy-lower alkyl, lower alkoxy-carbonyl, CN, NO₂, N-heterocyclyl or N-heterocyclyl-lower alkyl, or optionally mono- or di-lower alkyl substituted amino;
R¹² is is independently H, or optionally substituted (lower alkyl, aryl, aryl-lower alkyl, C₃-C₁₀cycloalkyl, C₃-C₁₀cycloalkyl-lower alkyl, heterocyclyl or heterocyclyl-lower alkyl), and
wherein R² is optionally substituted by halo, hydroxy, oxo, lower alkoxy, CN, NO₂, or optionally mono- or di-lower alkyl substituted amino.

Halo or halogen denote I, Br, Cl or F.

The term “lower” referred to above and hereinafter in connection with organic radicals or compounds respectively defines such as branched or unbranched with up to and including 7, preferably up to and including 5 and advantageously one, two or three carbon atoms. A lower alkyl group is branched or unbranched and contains 1 to 7 carbon atoms, preferably 1-5 carbon atoms. Lower alkyl represents; for example, methyl, ethyl, propyl, butyl, isopropyl isobutyl, tertiary butyl or neopentyl (2,2-dimethylpropyl).

Halo-substituted lower alkyl is C₁-C₇lower alkyl substituted by up to 6 halo atoms.

A lower alkoxy group is branched or unbranched and contains 1 to 7 carbon atoms, preferably 1-4 carbon atoms. Lower alkoxy represents for example methoxy, ethoxy, propoxy, butoxy, isopropoxy, isobutoxy or tertiary butoxy.

A lower alkene, alkenyl or alkenyloxy group is branched or unbranched and contains 2 to 7 carbon atoms, preferably 2-4 carbon atoms and contains at least one carbon-carbon double bond. Lower alkene lower alkenyl or lower alkenyloxy represents for example vinyl, prop-1-enyl, allyl, butenyl, isopropenyl or isobutenyl and the oxy equivalents thereof.

A lower alkyne, alkynyl or alkynyloxy group is branched or unbranched and contains 2 to 7 carbon atoms, preferably 2-4 carbon atoms and contains at least one carbon-carbon triple bond. Lower alkyne or alkynyl represents for example ethynyl, prop-1-ynyl, propargyl, butynyl, isopropynyl or isobutynyl and the oxy equivalents thereof.

In the present description, oxygen containing substituents, e.g. alkoxy, alkenyloxy, alkynyloxy, carbonyl, etc. encompass their sulphur containing homologues, e.g. thioalkoxy, thioalkenyloxy, thioalkynyloxy, thiocarbonyl, sulphone, sulphoxide etc.

Aryl represents carbocyclic or heterocyclic aryl.

Carbocyclic aryl represents monocyclic, bicyclic or tricyclic aryl, for example phenyl or phenyl mono-, di- or tri-substituted by one, two or three radicals selected from lower alkyl, lower alkoxy, aryl, hydroxy, halogen, cyano, trifluoromethyl, lower alkylenedioxy and oxy-C₂-C₃-alkylene and other substituents, for instance as described in the examples; or 1- or 2-naphthyl; or 1- or 2-phenanthrenyl. Lower alkylenedioxy is a divalent substituent attached to two adjacent carbon atoms of phenyl, e.g. methylenedioxy or ethylenedioxy. Oxy-C₂-C₃-alkylene is also a divalent substituent attached to two adjacent carbon atoms of phenyl, e.g. oxyethylene or oxypropylene. An example for oxy-C₂-C₃-alkylene-phenyl is 2,3-dihydrobenzofuran-5-yl.

Preferred as carbocyclic aryl is naphthyl, phenyl or phenyl optionally substituted, for instance, as described in the examples, e.g. mono- or disubstituted by lower alkoxy, phenyl, halogen, lower alkyl or trifluoromethyl.

Heterocyclic aryl represents monocyclic or bicyclic heteroaryl, for example pyridyl, indolyl, quinoxalinyl, quinolinyl, isoquinolinyl, benzothienyl, benzofuranyl, benzopyranyl, benzothiopyranyl, furanyl, pyrrolyl, thiazolyl, oxazolyl, isoxazolyl, triazolyl, tetrazolyl, pyrazolyl, imidazolyl, thienyl, or any said radical substituted, especially mono- or di-substituted as defined above.

Preferably, heterocyclic aryl is pyridyl, indolyl, quinolinyl, pyrrolyl, thiazolyl, isoxazolyl, triazolyl, tetrazolyl, pyrazolyl, imidazolyl, thienyl, or any said radical substituted, especially mono- or di-substituted as defined above.

Cycloalkyl represents a saturated cyclic hydrocarbon optionally substituted by lower alkyl which contains 3 to 10 ring carbons and is advantageously cyclopropyl, cyclopentyl, cyclohexyl, cycloheptyl or cyclooctyl optionally substituted by lower alkyl.

N-heterocyclyl is as defined above. Preferred N-heterocyclic substituents are optionally substituted pyrrolidine, pyrrole, diazole, triazole, tetrazole, imidazole, oxazole, thiazole, pyridine, pyrimidine, triazine, piperidine, piperazine, morpholine, phthalimde, hydantoin, oxazolidinone or 2,6-dioxo-piperazine and, for example, as hereinafter described in the examples.

In a further embodiment the invention provides a compound of formula VIII, or a pharmaceutically acceptable salt or ester thereof 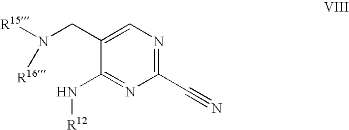
wherein R¹² is as defined above and R¹⁵′″ and R^{16′″} are as defined above for R¹⁵ and R¹⁶ respectively.

R¹² is preferably R¹²′″ which is lower alkyl, e.g. straight chain or more preferably branched-chain C₁-C₆ alkyl, e.g. especially 2-ethylbutyl, isobutyl, or 2,2-dimethylpropyl; or C₃-C₆cycloalkyl, especially cyclopropyl, cyclopentyl or cyclohexyl.

R¹⁵′″ and R¹⁶′″ may be such that R¹⁵′″ and R¹⁶′″ together with the nitrogen atom to which they are joined to form an N-heterocyclyl group. R¹⁵′″ is preferably optionally substituted (aryl-lower-alkyl, heterocyclyl-aryl, N-heterocyclyl-aryl or aryl-N-heterocyclyl (where N-heterocyclyl is as defined above). R¹⁵′″ is preferably optionally substituted by from 1-4 substituents selected from halo, hydroxy, nitro, cyano, lower-alkyl, lower-alkoxy or lower-alkoxy-lower-akyl. For example, R¹⁵′″ is 4-methoxy-benzyl, 3-methoxy-benzyl, 4-(4-methyl-piperazin-1-yl)-benzyl, 4-[4-(2-ethoxy-ethyl)-piperazin-1-yl]-benzyl, 1-methyl-1-phenyl-ethyl, 2-(4-methoxy-phenyl)-1,1-dimethyl-ethyl, 2-(4-fluoro-phenyl)-1,1-dimethyl-ethyl, 4-(4-methyl-piperazin-1-yl)-phenyl]-ethyl, 2-[4-(4-isopropyl-piperazin-1-yl)-phenyl]-1,1-dimethyl-ethyl, 2-{4-[4-(2-methoxy-ethyl)-piperazin-1-yl]-phenyl}-1,1-dimethyl-ethyl, 2-{3-[4-(2-ethoxy-ethyl)-piperazin-1yl-]-phenyl}-1,1-dimethyl-ethyl, 2-[3-(4-ethyl-piperazin-1-yl)-phenyl]-1,1-dimethyl-ethyl, 2-[3-(4-isopropyl-piperazin-1-yl)-phenyl]-1,1-dimethyl-ethyl, 1,1-dimethyl-2-(3-pyrrolidin-1-yl-phenyl)-ethyl, 2-{3-[4-(2-methoxy-ethyl)-piperazin-1-yl]-phenyl}-1,1-dimethyl-ethyl, 2-(4-metoxy-phenyl)-ethyl, 2-[4-(4-methyl-piperazin-1-yl)-phenyl]-ethyl, 2-[4-(4-isopropyl-piperazin-1-yl)-phenyl]-ethyl, 2-{4-[4-(2-methoxy-ethyl)-piperazin-1-yl]-phenyl}-ethyl, 2-(3-methoxy-phenyl)-ethyl, 2-[3-(4-methyl-piperazin-1-yl)-phenyl]-ethyl, 2-[4-(4-isopropyl-piperazin-1-yl)-phenyl]-ethyl, 2-pyrrol-1-yl-ethyl, 3-piperidin-1-yl-propyl, 2-(4-methoxy-phenyl)-2-methyl-propyl, 2-methyl-2-[4-(4-methyl-piperazin-1-yl)-phenyl]-propyl, 2-[4-(4-isopropyl-piperazin-1-yl)-phenyl]-2-methyl-propyl, 2-{4-[4-(2-ethoxy-ethyl)-piperazin-1-yl]-phenyl}-2-methyl-propyl, 2-{4-[pyrimidin-1-yl]-phenyl}-2-methyl-propyl, 4-(3-methoxy-phenyl)-piperazin-1-yl-methyl, 4-(4-methoxy-phenyl)-piperazin-1-yl-methyl, 1-methyl-1-(1-phenyl-cyclopropyl)-ethyl. For example, R¹⁵′″ and R¹⁶′″ together with the nitrogen atom to which they are joined to form an N-heterocyclyl group is 4-(2-pyridin-4-yl-ethyl)-piperazin-1-yl, [4-(2-pyridin-2-yl-ethyl)-piperazin-1-yl, 4-pyridin-4-ylmethyl-piperazin-1-yl, 4-(2-piperidin-1-yl-ethyl)-piperazin-1-yl, 4-(2-pyrrolidin-1-yl-ethyl)-piperazin-1-yl, 4-(2-Diethylamino-ethyl)-piperazin-1-yl, 4-(3-Diethylamino-propyl)-piperazin-1-yl, 4-(1-methyl-piperidin-4-yl)-piperazin-1-yl, 4-pyrrolidin-1-yl-piperidin-1-yl, 4-(2-methoxy-ethyl)-piperazin-1-yl.

In a preferred embodiment the invention provides the use according to the invention of a compound of formula IX, or a pharmaceutically acceptable salt or ester thereof 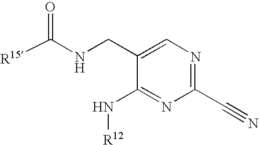
wherein R¹² is as defined above and R^{15′} is as defined above for R¹⁵.

R¹² is preferably R¹²′ which is lower alkyl, e.g. straight chain or more preferably branched-chain C₁-C₆ alkyl, e.g. especially 2-ethylbutyl, isobutyl, or 2,2-dimethylpropyl; or C₃-C₆cycloalkyl, especially cyclopropyl, cyclopentyl or cyclohexyl.

R¹⁵′ is preferably optionally substituted (aryl-lower-alkyl, heterocyclyl-aryl, N-heterocyclyl-aryl or aryl-N-heterocyclyl (where N-heterocyclyl is as defined above). R¹⁵′ is preferably optionally substituted by from 1-4 substituents selected from halo, hydroxy, nitro, cyano, lower-alkyl, lower-alkoxy, lower-alkoxy-carbonyl or lower-alkoxy-lower-akyl. For example, R¹⁵′ is 4-methoxy-phenyl, 4-(1-propyl-piperidin-4-yl)-phenyl, 4-(4-methyl-piperazin-1-yl)-phenyl, 4-[1-(2-methoxy-ethyl)-piperidin-4-yl]-phenyl, 4-(4-propyl-piperazin-1-yl)-phenyl, 3-[4-(4-methyl-piperazin-1-yl)-phenyl]-propionyl, 3-[3-(4-methyl-piperazin-1-yl)-phenyl]-propionyl, 4-(4-ethyl-piperazin-1-yl)-phenyl, 4-(4-isopropyl-piperazin-1-yl)-phenyl, 4-[4-(2-ethoxy-ethyl)-piperazin-1-yl]-phenyl, 4-[4-(2-methoxy-ethyl)-piperazin-1-yl]-phenyl, 4-piperazin-1-yl-phenyl, 4-[4-(carboxylic acid tert-butyl ester) piperazino-1-yl-]-phenyl, 3-[4-(carboxylic acid tert-butyl ester) piperazino-1-yl-]-phenyl, 3-(4-methyl-piperazin-1-yl)-phenyl, 3-(4-ethyl-piperazin-1-yl)-phenyl, 3-(4-isopropyl-piperazin-1-yl)-phenyl, 3-[4-(2-methoxy-ethyl)-piperazin-1-yl]-phenyl, 3-[4-(2-ethoxy-ethyl)-piperazin-1-yl]-phenyl, 3-(2-pyrrolidin-1-yl-ethoxy)-phenyl, 3-(2-dimethylamino-ethoxy)-4-methoxy-phenyl, 4-dimethylaminomethyl-phenyl, 4-(4-methyl-piperazin-1-ylmethyl)-phenyl, 4-[1-(2-methoxy-ethyl)-piperidin-4-ylmethyl]-phenyl, 4-methoxy-3-(2-piperidin-1-yl-ethoxy)-phenyl, 3-[4-(4-ethyl-piperazin-1-yl)-phenyl]-2,2-dimethyl-propionyl, 3-[4-(4-propyl-piperazin-1-yl)-phenyl]-propionyl, 3-(4-pyrrolidin-1-yl-phenyl)-propionyl, 3-[3(4-ethyl-piperazin-1-yl)-phenyl]-2,2-dimethyl-propionyl, 3-{3-[4-(2-methoxy-ethyl)-piperazin-1-yl]-phenyl}-2,2-dimethyl-propionyl, 3-{3-[4-(2-ethoxy-ethyl)-piperazin-1-yl]-phenyl}-2,2-dimethyl-propionyl, 3-(3-pyrrolidin-1-yl-phenyl)-propionyl, 2-[4-(4-methyl-piperazin-1-yl)-phenyl]-isobutyl, 2-(4-methoxy-phenyl)-acetyl, 2-(3-methoxy-phenyl)-acetyl, 2-[4-(4-methyl-piperazin-1-yl)-phenyl]-acetyl, 2-[4-(4-ethyl-piperazin-1-yl)-phenyl]-acetyl, 2-[4-(4-isopropyl-piperazin-1-yl)-phenyl]-acetyl, 2-(4-pyrrolidin-1-yl-phenyl)-acetyl, 2-[4-(2-diethylamino-ethylamino)-phenyl]-isobutyl, 2-(4-pyrrolidin-1-yl-phenyl)-isobutyl.

Particularly preferred compounds are examples as disclosed in WO 03/020278A1, pp. 17-52.

All the cathepsin K inhibitors mentioned above as an alternative class of cathepsin K compounds for use in the invention are known from the literature. This includes their production (see e.g. WO 03/020278A1, pp. 9-12).

The cathepsin K inhbitors may be administered as the sole active ingredient or in conjunction with, e.g. as an adjuvant to, another therapeutic agent (Other Agent). Examples of Other Agents include, but are not limited to, agents useful for treating or preventing a bone-resorbing disease, a neoplastic disease, arthritis, a disease exacerbated by the presence of a high cathepsin K activity or a disease improved by the presence of a cathepsin K inhibitor; activating the function of cathepsin K in a bone cell; inhibiting the function of cathepsin K in a cancer cell; inhibiting the expression of cathepsin K in a cell; and inhibiting the growth of a neoplastic cell. The Other Agent can be administered before, after or concurrently with the cathepsin K inhibitors. In these embodiments, the time at which the cathepsin K inhibitors exerts their therapeutic effect on the patient overlaps with the time at which the Other Agent exerts its therapeutic effect on the patient.

In one embodiment, the Other Agent is useful for the treatment or prevention of a bone-loss disease (e.g., osteoporosis). Other Agents useful for the treatment or prevention of a bone-loss disease include, but are not limited to, other cathepsin K inhibitors than the first cathepsin K inhibitor (see below for examples), bisphosphonates (e.g., eitodronate, pamidronate, alendronate, risedronate, zoledronic acid, ibandronate, clodronate or tiludronate), Selective Estrogen Receptor Modulators (SERMs), such as tamoxifen, raloxifene, medroxyprogesterone, danizol and gestrinone, parathryoid hormone (“PTH”) or fragments or analogs thereof, compounds that release endogenous PTH (e.g., a PTH releasing compounds) and calcitonin or fragments or analogs thereof.

In another embodiment, the Other Agent is useful for the treatment or prevention of a neoplastic disease. In one embodiment, the other therapeutic agent is useful for the treatment or prevention of cancer (e.g., cancer of the breast, ovary, uterine, prostate or hypothalamus). Other therapeutic agents useful for the treatment or prevention of cancer or a neoplastic disease include, but are not limited to, alkylating agents (e.g., nitrosoureas), an anti-metabolite (e.g. methotrexate or hydroxyurea), etoposides, campathecins, bleomycin, doxorubicin, daunorubicin, colchicine, irinotecan, camptothecin, cyclophosphamide, 5-fluorouracil, cisplatinum, carboplatin, methotrexate, trimetrexate, erbitux, thalidomide, taxol, a vinca alkaloid (e.g., vinblastine or vincristine) or a microtubule stabilizer (e.g., an epothilone).

Further illustrative examples of Other Agents useful for the treatment or prevention of cancer include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; ImiDs; interleukin II (including recombinant interleukin II, or rIL2), interferon -2a; interferon alpha-2b; interferon alpha-n1; interferon alpha-n3; interferon beta-Ia; interferon gamma-Ib; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine, mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; SelCid; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; temozolomide; temodar; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other Agents useful for the treatment or prevention of cancer include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzopyranones, benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); cell-cycle inhibitors (e.g., flavopiridol A, tryprostatin B, p19ink4D); cyclin-dependent kinase inhibitors (e.g., roscovitine, olomucine and purine analogs); MAP kinase inhibitors (CNI-1493); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor, multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; retinoic acid (e.g., 9-cis RA); histone deacetylase inhibitors (e.g., sodium butyrate, suberoylanilide hydroxamic acid); TRAIL; ras famesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred additional anti-cancer drugs are 5-fluorouracil and leucovorin.

In accordance with the foregoing the present invention provides in a yet further aspect:

A method as defined above comprising co-administration, e.g. concomitantly or in sequence, of a therapeutically effective amount of a cathepsin K inhibitor, and at least one second drug substance, said second drug substance being a therapeutic agent against bone loss diseases, e.g. as indicated above.

Or, a therapeutic combination, e.g. a kit (=packaging), comprising of a therapeutically effective amount of a) a cathepsin K inhibitor, and b) at least one second substance selected from a therapeutic agent against bone loss diseases, e.g. as indicated above. The kit may comprise instructions for its administration.

Where the cathepsin K inhibitors are administered in conjunction with other therapeutic agents against bone loss diseases, dosages of the co-administered combination compound will of course vary depending on the type of co-drug employed, e.g. whether it is a bisphosphonate, a SERMs, a calcitonin, a PTH, a PTH fragment or a PTH analogue or others, on the specific drug employed, on the condition being treated and so forth. Pharmaceutical compositions comprising cathepsin K inhibitors and a second drug substance may be manufactured in conventional manner. A composition according to the invention may be administered by any conventional route, for example parenterally, e.g. in the form of injectable solutions (e.g. for zoledronic acid) or suspensions, or enterally, preferably orally (e.g. for Compound A, see Example 1), e.g. in tablets or capsules.

A “cathepsin K inhibitor” is a compound that binds to and inhibits the function of cathepsin K in one or more cells or tissues. Cathepsin K is e.g. disclosed in Tetzuka et al., 1994, J Biol Chem 269: 1106-1109 and includes isoforms or mutations of it, and a protein having at least 95% homology to cathepsin K.

The term “effective amount” in connection with a cathepsin K inhibitor means an amount capable of treating a bone loss disease, in particular severe bone loss diseases, preferably severe osteoporosis, preferably severe osteoporosis in postmenopausal women, a neoplastic disease, arthritis, a disease exacerbated by the presence of cathepsin K activity or a disease improved by the presence of cathepsin K inhibitors; activating the function of cathepsin K in a bone cell; inhibiting the function of cathepsin K in a cancer cell; inhibiting the expression of cathepsin K in a cell; or inhibiting the growth of a neoplastic cell.

The term “effective amount” in connection with another therapeutic agent means an amount capable of treating or preventing a bone loss disease, in particular severe bone loss diseases, preferably severe osteoporosis, preferably severe osteoporosis in postmenopausal women, a neoplastic disease, arthritis, a disease exacerbated by the presence of estrogen or a disease improved by the presence of cathepsin K inhibitors; activating the function of cathepsin K in a bone cell; inhibiting the function of cathepsin K in a cancer cell; inhibiting the expression of cathepsin K in a cell; or inhibiting the growth of a neoplastic cell, while the cathepsin K inhibitor is exerting its therapeutic or prophylactic effect.

The term “a severe form of bone loss diseases” means one severe form of bone loss diseases as defined above or can mean several severe forms of bone loss diseases.

The term “severe osteoporosis” is to be understood according to WHO, i.e. severe osteoporosis is considered to be present when the value for bone mineral content is more than 2.5 SDs below the mean for young adults and there is at least one so-called fragility fracture (a fracture assumed to be associated with osteoporosis because it occurred as a result of slight trauma).

The term “bone-mineral density” or BMD means that the amount of mineral in a specific area of bone is measured. The more mineral, the denser the bone. Mineral is measured in grams; area is measured in square centimeters—and BMD is described as grams per square centimeter.

The term “T-score” compares the bone density with that of the average healthy young adult woman at the age of 35. T-scores are based on a statistical measure called the standard deviation (SD), which reflects differences from the average score.

A “patient” is an animal, including, but not limited to, an animal such a cow, monkey, horse, sheep, pig, chicken, turkey, quail, cat, dog, mouse, rat, rabbit, and guinea pig, in one embodiment a mammal, in another embodiment a human.

The invention is further described by way of illustration in the following Examples.

### EXAMPLE

### Example 1

### N-[1-(cyanomethyl-carbamoyl)-cyclohexyl]-4-(4-propyl-piperazin-1-yl)-benzamide (Compound A) positive effects on bone mineral density (BMD) and biomechanics in ovariectomized (OVX) Cynomolgus Monkeys after daily oral treatment for 18 months

The present, 18 month long study is performed in order to assess the effect of COMPOUND A on bone in a non-human primate model of osteoporosis. The OVX cynomolgus monkey is chosen as it has been shown in several studies to exhibit osteopenia and reduced bone strength (Jermoe C P, Peterson PE (2001) Bone; 29 (1): 1-6).

Methods

One hundred purpose-bred female cynomolgus monkeys (Macaca fascicularis), 12-13 years of age with closed growth plates are used for this study conducted in compliance with GLP. Eighty animals underwent bilateral ovariectomy and monkeys assigned to the sham group (S) underwent sham surgery. They are dosed twice daily by oral gavage with vehicle (distilled water) or COMPOUND A maleate (COMPOUND A-AF) for 18 months (Table 1).

TABLE 1
Treatment groups

| | | | Dose | Dose | Number |
|---|---|---|---|---|---|
| | OVX | | (mg/kg/day) | (mg/kg/day) | of |
| Group | Status | Test Article | 1st month | months 2-18 | Animals |
| S | Sham | Vehicle | 0 | 0 | 20 |
| O | OVX | Vehicle | 0 | 0 | 20 |
| L | OVX | COMPOUND | 2 × 3 | 2 × 3 | 20 |
| | | A-AF | | | |
| M | OVX | COMPOUND | 2 × 10 | 2 × 10 | 20 |
| | | A-AF | | | |
| H | OVX | COMPOUND | 2 × 50 | 2 × 30 | 20 |
| | | A-AF | | | |

DXA (Dual Energy X-ray Absorptiometry) of the lumbar spine and femur is performed twice prior to and at 3 months intervals during treatment. Compression test of third lumbar vertebra and a three-point bending test for the midshaft femur are carried out according to standard procedures. In brief, the cranial and caudal ends of each vertebral body are cut off to obtain a vertebral body specimen with two parallel surfaces and a height of approximately 7 mm. Each specimen is placed between two plates and a load applied at a constant displacement rate of 6 mm/min until failure in an Instron Mechanical Testing Machine. The femur is placed on the lower supports of a three point bending fixture with the anterior side facing downward in an Instron Mechanical Testing Machine. Load is applied at a constant displacement rate of 12 mn/min until failure.

All group data are first checked to ensure they meet the assumptions for parametric analysis (normality, homogeneity of variances). Data are transformed, if necessary, to meet the assumptions as closely as possible. Results using transformed data are used for interpretation. In general, data are analyzed by one-way analysis of variance (1-way ANOVA) for variables assessed only once during the treatment phase of the study. For variables assessed repeatedly during the treatment phase, a two-way (group, time) analysis of covariance (ANCOVA) with repeated measures on time is used. For each ANCOVA evaluation, average baseline data for individual animals is used as the covariate. Results are presented as Mean±SEM (standard error of mean).

Results

COMPOUND A is generally well tolerated.

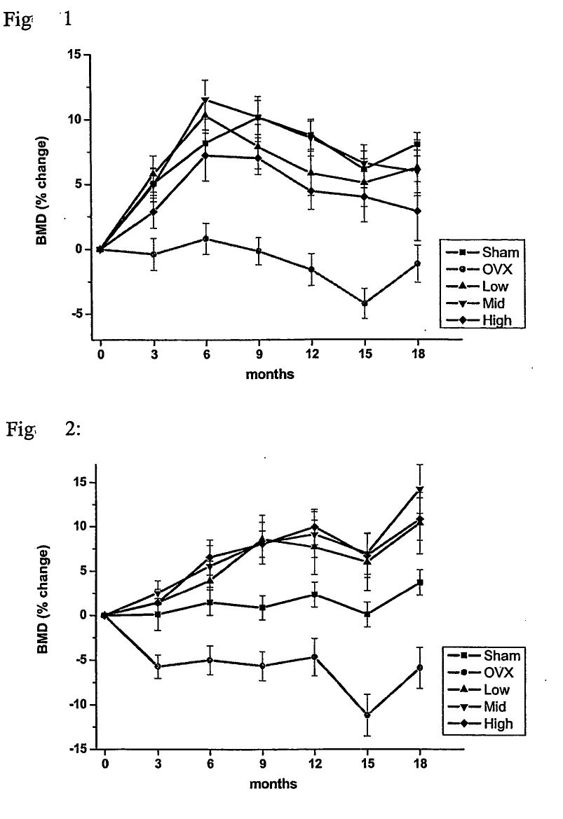

Baseline bone mineral density (BMD) of lumbar vertebrae (LV) 1-4 is not significantly different between groups. LV BMD increased in group S until months 6-9 and remained stable thereafter (FIG. 1). In contrast, LV BMD does not change in group O and is significantly lower than in group S from month 3 until the end of the study.

All three doses of COMPOUND A inhibit the effect of OVX on lumbar spine LV1-4 BMD. Group H tended to be less efficacious, which may be explained by its effect on food consumption and body weight gain.

See FIG. 1: Lumbar spine BMD (percent changes), Percent changes from baseline of lumbar vertebrae 1-4 BMD; means±SEM, n=19-20; p<0.05 versus OVX for all groups by repeated measures analysis.

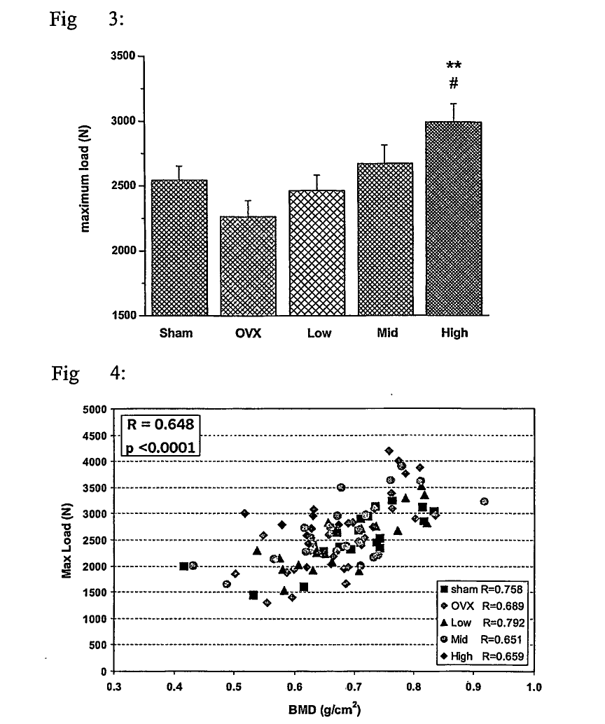

In contrast to the vertebrae, BMD of the femur does not increase over time in group S animals (FIG. 2) and OVX caused a significant decrease. All three dose levels of COMPOUND A cause a significant increase in whole femur BMD compared to group O over the whole 18 months period (FIG. 2). This is most pronounced for proximal and distal femur (not shown), but also seen in the midshaft femur.

Whole femur BMD values of COMPOUND A-treated groups are even above sham for most time points, and differences for absolute values are significant at month 9 for groups L and H and at month 18 for group M. The anterior-posterior diameter of the femur midshaft tends to be larger in groups L and H than in groups S and O (Table 3).

See FIG. 2: Whole femur BMD (percent changes)Percent change from baseline of whole femur BMD; means±SEM, n=19-20; p<0.05 versus OVX for all groups and time points except group Sham at 12 months.

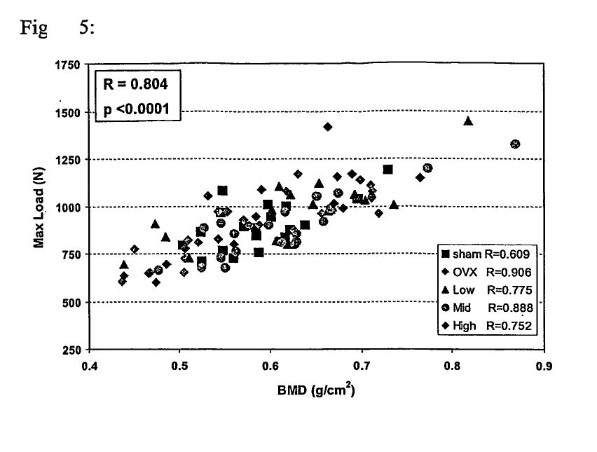

Biomechanical testing of lumbar vertebrae 3 (LV3) demonstrates higher values of maximum load for group S as compared to group O, but difference are not statistically significant (FIG. 3). All COMPOUND A treatment groups increase maximum load and the effect is significant for group H. Maximum load is even increased above the group S level for this group. A highly significant correlation between BMD and maximum load is obtained for all groups (FIG. 4).

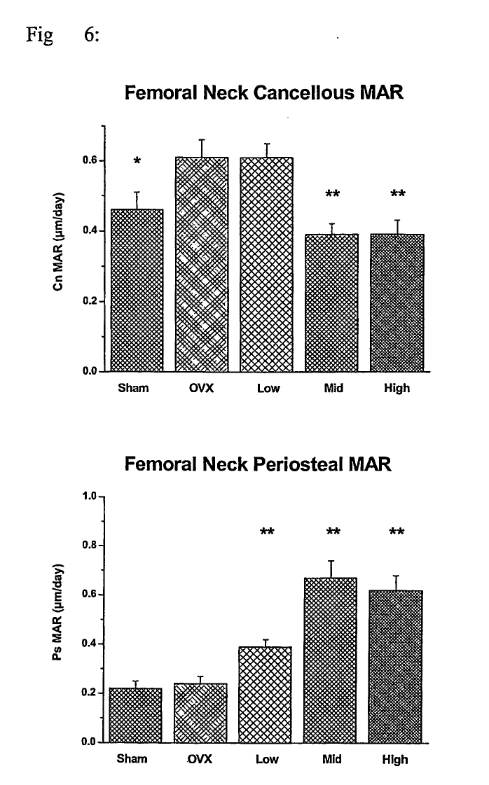

For the 3-point bending test of the left femur midshaft, similar results are obtained for group S and group O indicating that ovariectomy does not have a significant impact on the mechanical properties (Table 2). Values for groups L, M, and H are higher than group O, although statistically significant only for energy and toughness for groups L and H. Maximum load and BMD are highly significant correlated for all groups (FIG. 5).

See FIG. 3: Lumbar vertebrae maximum load—Maximum load in LV3 compression test; means±SEM, n=19-20; ** p<0.01 versus OVX, # p<0.05 versus Sham.

See FIG. 4: Lumbar vertebrae BMD versus maximum load—Correlation of LV3 maximum load with LV3 BMD at 18 months; p<0.01 for all groups.

TABLE 2
Midshaft femur biomechanics

| | Sham | OVX | Low | Mid | High |
|---|---|---|---|---|---|
| Max load (N) | 897 ± 28 | 850 ± 44 | 972 ± 38 | 894 ± 39 | 973 ± 45 |
| Energy (mJ) | 1075 ± 71 | 974 ± 75 | 1305 ± 91 * | 1126 ± 88 | 1276 ± 87 * |
| Ultimate strength | 195 ± 3 | 186 ± 5 | 195 ± 5 | 193 ± 3 | 196 ± 4 |
| (N/mm²) | | | | | |
| Toughness (MJ/mm³) | 3.2 ± 0.2 | 2.9 ± 0.2 | 3.7 ± 0.2 * | 3.3 ± 0.2 | 3.6 ± 0.2 * |
| Moment of inertia | 286 ± 11 | 282 ± 15 | 322 ± 20 | 290 ± 16 | 322 ± 20 |
| (mm⁴) | | | | | |

3-point bending test of midshaft femur; means ± SEM, n = 19-20;
* and bold = p < 0.05 versus OVX

See FIG. 5: Midshaft femur BMD versus maximum load—Correlation of midshaft femur maximum load with midshaft femur BMD, as measured by in vivo DXA at 18 months; p<0.01 for all groups.

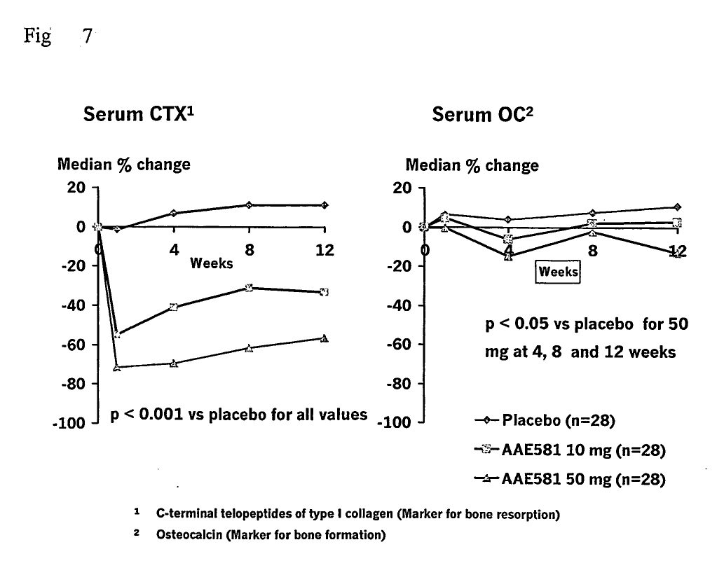

See FIG. 6: Mineral Apposition Rate (MAR)

Mineral apposition rate (MAR), an indicator of bone formation, is reduced at cancellous bone for the femoral neck by the mid and high dose (FIG. 6) consistent with the action of an inhibitor of bone turnover. Unexpectedly, MAR is however highly significantly increased at the periosteal side of the femoral neck and even the low dose is active at this site (for measurement of the MAR: see Parfitt A M et al., J. Bone Miner Res 1987; 2: 595-610).

Summary:

Ovariectomized (OVX) cynomolgus monkeys are treated orally for 18 months with 3, 10, or 50/30 mg/kg COMPOUND A maleate twice daily (bid). COMPOUND A treatment is well tolerated at 3 and 10 mg/kg bid. The 50 mg/kg bid dose leads to decreases in food intake and body weight so that it is reduced to 30 mg/kg bid after one month. Body weight gain recovered but remains significantly lower until the end of the study which may have influenced bone parameters.

OVX animals have significantly lower BMD at lumbar vertebrae LV1-4 (−7%) and the whole femur (−7.7%) than sham-operated ones as measured by DXA after 18 months. While OVX causes a decrease of the femur BMD from baseline, it prevents the increase of BMD in vertebrae seen in sham animals. Bone strength is reduced in parallel to BMD although significant differences are neither seen in lumbar vertebrae (compression test) nor in the femur midshaft (3-point bending test).

All three dose groups of COMPOUND A are effective in inhibiting the effect of OVX on LV1-4 BMD. They also cause a significant increase in whole femur BMD compared to the OVX group over the whole 18 months period. This is most pronounced for proximal and distal femur, but also seen in the femur midshaft. Unexpectedly, whole femur BMD values of COMPOUND A-treated groups are even above the sham group for most time points. Changes in bone mineral content parallel those of BMD. Compared to OVX controls, COMPOUND A treatment increases bone strength in lumbar vertebrae and the femur midshaft, although not all differences in biomechanical parameters reach statistical significance. However, in vertebrae and femur BMD and strength (maximum load) are highly significantly correlated in individual animals of control as well as COMPOUND A-treated groups at all three dose levels.

In conclusion, COMPOUND A prevents the negative effects of OVX on spinal and femoral BMD and bone strength. At the latter site it causes even a BMD increase above the sham-operated animals. BMD is significantly correlated with bone strength indicating normal bone quality in COMPOUND A-treated animals. Bone formation is increased at peristoeal sites, while it is decreased at cancellous bone.

EXAMPLE 2
Compound A has a potent and rapid action on bone resorption
marker (sCTX1)
a) Composition of placebo and Compound A comprising hard gelatin
capsules (mg)

| | Placebo | 5 mg | 25 mg | 50 mg |
|---|---|---|---|---|
| Compound A | — | ⁽¹⁾6.41 | ⁽²⁾32.05 | ⁽³⁾64.1 |
| Lactose | 210.6 | 276.2 | 250.55 | 218.5 |
| Starch | 144.0 | — | — | — |
| Pregelatinized starch | — | 72.0 | 72.0 | 72.0 |
| Colloidal anhydrous silica | 1.8 | 1.8 | 1.8 | 1.8 |
| Magnesium stearate | 3.6 | 3.6 | 3.6 | 3.6 |
| Total weight of capsule fill | 360.0 | 360.0 | 360.0 | 360.0 |

⁽¹⁾corresponding to 5 mg free base
⁽²⁾corresponding to 25 mg free base
⁽³⁾corresponding to 50 mg free base

In a 12-week treatment, multicenter, double-blind, randomized, placebo-controlled, parallel group, a dose-ranging, safety, tolerability and efficacy trial with Compound A in postmenopausal women, with 3 weeks follow-up is conducted.

The primary objectives of the study are to assess the effect of Compound A on biochemical markers of bone resorption and bone formation, and to evaluate its safety and tolerability profile. Secondary objectives are to assess the changes in biochemical markers after the end of treatment, and to study the pharmacokinetics of Compound A and its metabolite during and after 12 weeks of treatment.

The population of subjects is normal healthy postmenopausal women. The reason for not investigating osteopenic women is the following: The efficacy endpoints of the study are biochemical markers of bone turnover. These variables are not directly correlated with BMD in man. Therefore we do not need to assess BMD and can include normal postmenopausal women. They are at least 5 years postinenopause, mainly because biomarkers are expected to fluctuate less in these women than in the perimenopause. Since the subjects included in the trial will not have any benefit whatsoever, and since the trial is rather demanding with a large number of assessments, including occult blood in stool, and PK.

Four doses are tested, 5, 10, 25, and 50 mg od. The duration of the study is 12 weeks, with a 3-week follow-up, 12 weeks treatment allows to assess the timecourse of biomarkers of both, bone resorption and bone formation and to ascertain that a steady-state in biomarkers is achieved.

Results:

See FIG. 7: Compound A has a potent and rapid action on bone resorption marker (sCTX1) without much affecting bone formation markers
140 postmenopausal women (28 subjects in all groups)
Double-blind, placebo controlled phase IIA study
All doses (except 5 mg) showed a significant difference (p<0.001) versus placebo at all time points (data for 5 mg and 25 mg not shown)
Dose-response relationship vs. placebo at all time points
Results from other resorption biomarkers (serum NTX, urinary NTX) support results seen with serum CTX
For bone formation markers (serum osteocalcin, BSAP) over time, the decrease of suppression is less than that seen with the bone resorption markers
Summary: Results suggest that bone resorption is prevented without affecting bone growth

1. A method for the treatment of a severe form of bone loss diseases in a patient in need of such treatment which comprises administering an effective amount of a c

2. The use of a cathepsin K inhibitor in the preparation of a medicament for the treatment of a severe form of bone loss diseases.

3. A pharmaceutical composition which incorporates as an active agent a cathepsin K inhibitor for use in the treatment of a severe form of bone loss diseases.

4. A method, use or composition according to claim 1, wherein the cathepsin K inhibitors are used to stimulate bone growth in a patient in need of such a treatment.

5. A method, use or composition according to claim 1, wherein the diseases are a severe form of osteoporosis, osteoarthritis or bone metastasis.

6. A method, use or composition according to claim 1, wherein the disease is severe osteoporosis.

7. A method, use or composition according to claim 1, wherein the disease is severe osteoporosis in postmenopausal women.

8. A method, use or composition according to claim 1, in which the cathepsin K inhibitor is selected from the following compounds of formula V or a pharmaceutically acceptable salt thereof, or any hydrate thereof 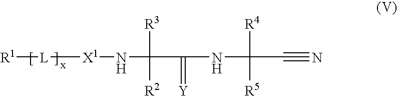
wherein
R¹ is optionally substituted (aryl, aryl-lower alkyl, lower alkenyl, lower alkynyl, heterocyclyl or heterocyclyl-lower alkyl);
R² and R³ together represent lower alkylene, optionally interrupted by O, S or NR⁶, so as to form a ring with the carbon atom to which they are attached, and R⁶ is hydrogen, lower alkyl or aryl-lower alkyl;
R⁴ and R⁵ are independently H, or optionally substituted (lower alkyl or aryl-lower alkyl), —C(O)OR⁷, or —C(O)NR⁷R⁸, wherein R⁷ is optionally substituted (lower alkyl, aryl, aryl-lower alkyl, cycloalkyl, bicycloalkyl, bicycloalkyl or heterocyclyl), and R⁸ is H, or optionally substituted (lower alkyl, aryl, aryl-lower alkyl, cycloalkyl, bicycloalkyl, bicycloalkyl or heterocyclyl); or
R⁴ and R⁵ together represent lower alkylene, optionally interrupted by O, S or NR⁶, so as to form a ring with the carbon atom to which they are attached, and R⁶ is hydrogen, lower alkyl or aryl-lower alkyl; or
R⁴ is H or optionally substituted lower alkyl and R⁵ is a substituent of formula —X²—(Y¹)ₙ—(Ar)ₚ-Q-Z
wherein
Y¹ is O, S, SO, SO₂, N(R⁶)SO₂, N—R⁶, SO₂NR⁶, CONR⁶ or NR⁶CO;
N is zero or one;
P is zero or one;
X² is lower alkylene: or when n is zero, X² is also C₂-C₇-alkylene interrupted by O, S, SO, SO₂, NR⁶, SO₂NR⁶, CONR⁶ or NR⁶CO, and R⁶ is hydrogen, lower alkyl or aryl-lower alkyl;
Ar is arylene;
Z is hydroxyl, acyloxy, carboxyl, esterified carboxyl, amidated carboxyl, aminosulfonyl, (lower alkyl or aryl-lower alkyl)aminosulfonyl, or (lower alkyl or aryl-lower alkyl)sufonylaminocarbonyl;
or Z is tetrazolyl, triazolyl or imidazolyl;
Q is a direct bond, lower alkylene, Y¹-lower alkylene or C₂-C₇-alkylene interrupted by Y¹;
X¹ is —C(O)—, —C(S)—, —S(O)—, —S(O)₂—, or —P(O)(OR⁶)—, and R⁶ is as defined above;
Y is oxygen or sulphur;
L is optionally substituted -Het-, -Het-CH₂— or —CH₂-Het-, and Het is a hetero atom selected from O, N or S; and
X is zero or one; and
aryl in the above definitions represents carbocyclic or heterocyclic aryl.

9. A method, use or composition according to claim 1, in which the cathepsin K inhibitor is N-[1-(cyanomethyl-carbamoyl)-cyclohexyl]4-(4-propyl-piperazin-1-yl)-benzamide, or a pharmaceutically acceptable salt thereof, e.g. the maleate form, or any hydrate thereof.

10. A pharmaceutical composition comprising less than 50.1 mg N-[1-(cyanomethyl-carbamoyl)-cyclohexyl]4-(4-propyl-piperazin-1-yl)-benzamide or a pharmaceutically acceptable salt thereof wherein the amount of the base form is less than 50.1 mg.

11. The pharmaceutical composition according to claim 11 comprising less than 64.2 mg N-[1-(cyanomethyl-carbamoyl)-cyclohexyl]-4-(4-propyl-piperazin-1-yl)-benzamide maleate.

12. All novel compounds, processes, pharmaceutical compositions, methods and uses substantially as hereinbefore described with particular reference to the Examples.
